SEQUENCE LISTING (4 sequences)

SEQ ID NO: 1 - protein, 640 aa, Homo sapiens
MSERKEGRGKGKGKKKERGSGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCQPGFTGARCTENVPMKVQNQEKAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSERYVSAMTTPARMSPVDFHTPSSPKSPPSEMSPPVSSMTVSMPSMAVSPFMEEERPLLLVTPPRLREKKFDHHPQQFSSFHHNPAHDSNSLPASPLRIVEDEEYETTQEYEPAQEPVKKLANSRRAKRTKPNGHIANRLEVDSNTSSQSSNSESETEDERVGEDTPFLGIQNPLAASLEATPAFRLADSRTNPAGRFSTQEEIQARLSSVIANQDPIAV

SEQ ID NO: 2 - protein, 850 aa, Homo sapiens
MRQVCCSALPPPPLEKGRCSSYSDSSSSSSERSSSSSSSSSESGSSSRSSSNNSSISRPAAPPEPRPQQQPQPRSPAARRAAARSRAAAAGGMRRDPAPGFSMLLFGVSLACYSPSLKSVQDQAYKAPVVVEGKVQGLVPAGGSSSNSTREPPASGRVALVKVLDKWPLRSGGLQREQVISVGSCVPLERNQRYIFFLEPTEQPLVFKTAFAPLDTNGKNLKKEVGKILCTDCATRPKLKKMKSQTGQVGEKQSLKCEAAAGNPQPSYRWFKDGKELNRSRDIRIKYGNGRKNSRLQFNKVKVEDAGEYVCEAENILGKDTVRGRLYVNSVSTTLSSWSGHARKCNETAKSYCVNGGVCYYIEGINQLSCKCPNGFFGQRCLEKLPLRLYMPDPKQKAEELYQKRVLTITGICVALLVVGIVCVVAYCKTKKQRKQMHNHLRQNMCPAHQNRSLANGPSHPRLDPEEIQMADYISKNVPATDHVIRRETETTFSGSHSCSPSHHCSTATPTSSHRHESHTWSLERSESLTSDSQSGIMLSSVGTSKCNSPACVEARARRAAAYNLEERRRATAPPYHDSVDSLRDSPHSERYVSALTTPARLSPVDFHYSLATQVPTFEITSPNSAHAVSLPPAAPISYRLAEQQPLLRHPAPPGPGPGPGPGPGPGADMQRSYDSYYYPAAGPGPRRGTCALGGSLGSLPASPFRIPEDDEYETTQECAPPPPPRPRARGASRRTSAGPRRWRRSRLNGLAAQRARAARDSLSLSSGSGGGSASASDDDADDADGALAAESTPFLGLRGAHDALRSDSPPLCPAADSRTYYSLDSHSTRASSRHSRGPPPRAKQDSAPL

SEQ ID NO: 3 - protein, 720 aa, Homo sapiens
MSEGAAAASPPGAASAAAASAEEGTAAAAAAAAAGGGPDGGGEGAAEPPRELRCSDCIVWNRQQTWLCVVPLFIGFIGLGLSLMLLKWIVVGSVKEYVPTDLVDSKGMGQDPFFLSKPSSFPKAMETTTTTTSTTSPATPSAGGAASSRTPNRISTRLTTITRAPTRFPGHRVPIRASPRSTTARNTAAPATVPSTTAPFFSSSTLGSRPPVPGTPSTQAMPSWPTAAYATSSYLHDSTPSWTLSPFQDAASSSSSSSSSATTTTPETSTSPKFHTTTYSTERSEHFKPCRDKDLAYCLNDGECFVIETLTGSHKHCRCKEGYQGVRCDQFLPKTDSILSDPTDHLGIEFMESEEVYQRQVLSISCIIFGIVIVGMFCAAFYFKSKKQAKQIQEQLKVPQNGKSYSLKASSTMAKSENLVKSHVQLQNYSKVERHPVTALEKMMESSFVGPQSFPEVPSPDRGSQSVKHHRSLSSCCSPGQRSGMLHRNAFRRTPPSPRSRLGGIVGPAYQQLEESRIPDQDTIPCQGIEVRKTISHLPIQLWCVERPLDLKYSSSGLKTQRNTSINMQLPSRETNPYFNSLEQKDLVGYSSTRASSVPIIPSVGLEETCLQMPGISEVKSIKWCKNSYSADVVNVSIPVSDCLIAEQQEVKILLETVQEQIRILTDARRSEDYELASVETEDSASENTAFLPLSPTAKSEREAQFVLRNEIQRDSALTK

SEQ ID NO: 4 - protein, 115 aa, Homo sapiens
MPTDHEEPCGPSHKSFCLNGGLCYVIPTIPSPFCRCVENYTGARCEEVFLPGSSIQTKSNLFEAFVALAVLVTLIIGAFYFLCRKGHFQRASSVQYDINLVETSSTSAHHSHEQH

(19) United States
(12) Patent Application Publication
(10) Pub. No.: US 2024/0352422 A1
(43) Pub. Date: Oct. 24, 2024

(54) DIRECT REPROGRAMMING OF CELLS INTO CARDIAC PURKINJE-LIKE CELLS USING A UNIVERSAL SMALLMOLECULE COCKTAIL

(71) Applicant: UNIVERSITY OF HOUSTON SYSTEM, HOUSTON, TX (US)

(72) Inventors: Bradley K. McConnell, The Woodlands, TX (US); Robert J. Schwartz, Houston, TX (US); Nicole Prodan, Houston, TX (US)

(73) Assignee: UNIVERSITY OF HOUSTON SYSTEM, Houston, TX (US)

(21) Appl. No.: 18/684,668

(22) PCT Filed: Aug. 5, 2022
(86) PCT No.: PCT/US2022/039546

§ 371 Date: Feb. 17, 2024

Related U.S. Application Data
(60) Provisional application No. 63/234,399, filed on Aug. 18, 2021.

(51) Int. Cl. C12N 5/077

(52) U.S. Cl. CPC C12N 5/0657; C12N 2500/62; C12N 2501/11; C12N 2501/385; C12N 2501/71; C12N 2501/73; C12N 2501/81; C12N 2501/999; C12N 2506/45

(57) ABSTRACT

The present disclosure pertains to compositions suitable for use in differentiating cardiac progenitor cells to cells that resemble cardiac Purkinje cells. Additional embodiments pertain to methods of generating such differentiated cardiac cells by exposing cardiac progenitor cells to the compositions. The present disclosure also pertains to methods of treating or preventing a cardiovascular disease in a subject by administering the compositions or differentiated cardiac cells to the subject. Further embodiments pertain to methods of generating a cardiac tissue by exposing cardiac progenitor cells to a composition of the present disclosure and associating the cardiac progenitor cells with a tissue scaffold. The present disclosure also pertains to the use of the differentiated cardiac progenitor cells to assess the efficacy of one or more compounds in the treatment or prevention of a cardiovascular disease. The present disclosure also pertains to the differentiated cardiac cells and cardiac tissues that include them.

### CROSS-REFERENCE TO RELATED APPLICATIONS

The present application claims the benefit of U.S. Provisional Patent Application No. 63/234,399, filed on Aug. 18, 2021. The entirety of the aforementioned application is incorporated herein by reference.

### BACKGROUND

Current methods of treating or preventing cardiovascular diseases have numerous limitations. Embodiments of the present disclosure aim to address the aforementioned limitations.

### SUMMARY

In some embodiments, the present disclosure pertains to a composition that includes the following compounds: Rolipram, a derivative thereof, or a combination thereof; Forskolin, a derivative thereof, or a combination thereof; CHIR99021, a derivative thereof, or a combination thereof; SB431542, a derivative thereof, or a combination thereof; Valproic acid, a derivative thereof, or a combination thereof; RG108, a derivative thereof, or a combination thereof; Parnate, a derivative thereof, or a combination thereof; Resveratrol, a derivative thereof, or a combination thereof; Retinoic acid, a derivative thereof, or a combination thereof; and a Neuregulin protein, a derivative thereof, or a combination thereof. In some embodiments, the compositions of the present disclosure also include Sodium Nitroprusside, a derivative thereof, or a combination thereof. In some embodiments, the compositions of the present disclosure also include Epinephrine, a derivative thereof, or a combination thereof.

In some embodiments, the compositions of the present disclosure are suitable for use in differentiating cardiac progenitor cells to cells that resemble cardiac Purkinje cells. Additional embodiments of the present disclosure pertain to methods of generating differentiated cardiac cells by exposing cardiac progenitor cells to a composition of the present disclosure.

In some embodiments, the differentiated cardiac cells resemble cardiac Purkinje cells. In some embodiments, the differentiated cardiac cells are genetically, functionally, morphologically, and electrophysiologically similar to native cardiac Purkinje cells.

Further embodiments of the present disclosure pertain to methods of treating or preventing a cardiovascular disease in a subject by administering the compositions of the present disclosure to the subject. In some embodiments, the administration includes locally administering the composition to a cardiac tissue of the subject. In some embodiments, the cardiac tissue is near or at the ventricular myocardium. In some embodiments, the administering results in the differentiation of the cardiac progenitor cells to cardiac Purkinje cells in the subject.

Additional embodiments of the present disclosure pertain to methods of treating or preventing a cardiovascular disease in a subject by administering differentiated cardiac cells to a subject. In some embodiments, the differentiated cardiac cells are formed by exposing cardiac progenitor cells to the compositions of the present disclosure.

In some embodiments, the present disclosure pertains to methods of generating a cardiac tissue by exposing cardiac progenitor cells to a composition of the present disclosure and associating the cardiac progenitor cells with a tissue scaffold. Additional embodiments of the present disclosure pertain to methods of assessing the efficacy of one or more compounds in the treatment or prevention of a cardiovascular disease by exposing the one or more compounds to the differentiated cardiac cells of the present disclosure. Further embodiments of the present disclosure pertain to the differentiated cardiac cells of the present disclosure and cardiac tissues that include them.

### DESCRIPTION OF THE DRAWINGS

FIG. 1 illustrates a method of forming cardiac Purkinje-like cells and using the cells for various therapeutic and diagnostic applications.

FIGS. 2A-E illustrate a strategy for creating Purkinje-like cells. FIG. 2A illustrates a co-gene edition approach using CRISPR-Cas9 knock-in system, where an mCherry-IRES-Puromycin tag is added at the c-terminal of CNTN2 gene at the same time that a Blasticidin-Luciferase (Blast-Luc) tag is added to the ACTB gene to allow the selection of the gene edited cells prior to differentiation, and after Purkinje differentiation. FIG. 2B illustrates an overall scheme for differentiating CMs into cardiac Purkinje-like cells using a small molecule cocktail outlined in Example 1.2. (“PURK-cocktail”). In brief, the AC16-CM and iPSC were co-gene edited using a CRISPR-Cas9 Knock-in approach. The iPSCs were differentiated into myocytes (iPSC-CM). The AC16-CM and iPSC-CM were then treated with the PURK-cocktail and characterized by downstream transcriptome and functional analysis to determine if the differentiated cells were cardiac Purkinje-like. FIG. 2C provides PCR genotyping, which confirms that the cells were successfully gene edited using CRISPR-Cas9 at the CTNT2 and ACTB genes. FIGS. 2D and 2E show FACS analysis of control (FIG. 2D) and PURK-cocktail treated cells (FIG. 2E). The control (vehicle) treated cell population showed minimal expression of CNTN2-IRES-mCherry+ cells, whereas the PURK-cocktail treated cells showed a significant amount of CNTN2-IRES-mCherry+ cell population. Created using BioRender.com.

FIGS. 3A-L show morphology of Purkinje-like cells differentiated with the “PURK-cocktail” compared to control CMs. FIGS. 3A-C show AC16-CMs treated with vehicle at day-7 under 4×, 10×, 20× magnification, respectively. FIGS. 3D-F show AC16-Purkinje-like cells treated with the “PURK-cocktail” at day-7 under 4×, 10×, 20× magnification, respectively. FIGS. 3G-I show iPSC-CMs treated with vehicle at day-7 under 4×, 10×, 20× magnification, respectively. FIGS. 3J-L show iPSC-Purkinje-like cells treated with the “PURK-cocktail” at day-7 under 4×, 10×, and 20× magnification, respectively.

FIGS. 4A-R show evaluation of expression of key Purkinje markers in control vs PURK-cocktail treated cells. Immunocytochemistry and fluorescent microscopy showed that the control cells did not express key Purkinje markers [ETV1 (FIG. 4B), IRX3 (FIG. 4F), SCN5a (FIG. 4G) or PCP4 (FIG. 4N)] or the CRISPR-Cas9 Knock-in CNTN2-IRES-mCherry (FIGS. 4H and 4O). In contrast, the PURK-cocktail treated cells strongly expressed ETV1 (FIG. 4D), IRX3 (FIG. 4J), SCN5a (FIG. 4K) or PCP4 (FIG. 4Q)) and the CRISPR-Cas9 Knock-in CNTN2-IRES-mCherry reporter gene (FIGS. 4L and 4R). DAPI was used to stain the cells nuclei.

FIG. 5 shows qPCR data evaluating the expression of key Purkinje markers in control and PURK-cocktail treated cells. The PURK-cocktail treatment induced a different genetic profile, which resembled closely to that of native cardiac Purkinje cells on the both AC16-CM and iPSC-CM compared to the control. *,**,***,**** corresponds to p<0.05, 0.01, 0.001, and 0.0001 in a 2-Way ANOVA test. ns corresponds to “not significant”.

FIGS. 6A-E show RNA-seq and GO-analysis of FACS Sorted PURK-cocktail treated cells at distinct differentiation time points. FIG. 6A shows RNA-seq (Heat Map) showing the gene expression of PURK-cocktail treated cells, compared to vehicle-treated control cells, on day-4 (D4) and day-7 (D7) of differentiation. PURK-cocktail treatment induced a genetic profile that closely resembled that of native cardiac Purkinje cells as compared to the control CMs. At day-4 (D4), the PURK-cocktail starts to induce a cardiac Purkinje-like genetic profile which becomes even more robust by day-7 (D7) of differentiation. Additionally, it can be observed that the expression of atrial or ventricular CM genes is down-regulated over time in the PURK-cocktail treated cells, as the cells become more Purkinje-like. FIGS. 6B and 6C show GO-analyses showing over-expressed and down-expressed genes, respectively, on day-4 (D4) of differentiation of PURK-cocktail treated cells. FIGS. 6D and 6E show GO-analyses showing the over-expressed and down-expressed genes, respectively, on day-7 (D7) of differentiation of PURK-cocktail treated cells. The GO analyses collectively show the process of trans-differentiation from cardiomyocytes to cardiac Purkinje-like cells (PURK-cells).

FIGS. 7A-7F provide electrophysiology of PURK-cocktail treated cells. FIG. 7A provide optical activation maps showing an absence of electrical signal propagation in the control cells (left). The white arrow shows the direction of a uniform electrical signal in the PURK-cocktail treated cells (right). FIG. 7B shows the conduction velocity of the PURK-cocktail treated cells was determined to be significantly faster than control. FIG. 7C shows that PURK-cocktail cells cultured on the MEA on day-7. FIG. 7D shows voltage data from one channel of the MEA (top) with a zoomed-in view of the cellular response (bottom). FIG. 7E shows averaged spike waveform over all channels of the MEA in black with standard deviation of the signal shown in gray. FIG. 7F shows heatmaps of the voltage amplitude distribution from various timepoints across the entire MEA. The black star in the t=0 ms heatmap indicates the stimulation electrode location. Warmer colors (red, orange) indicate higher spike amplitudes. A Welch's T-Test was used to compare the conduction velocities. *** corresponds to p<0.001

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Cardiovascular disease (CVD) remains the leading cause of death globally. For instance, in the United States, CVD claims the life of a person every 36 seconds.

To date, there are no treatments that can ameliorate the underlying basis of CVDs: cardiomyocyte cell death and fibrotic tissue formation. For this reason, novel approaches to possibly cure CVD are increasingly needed.

Studies have shown that restoring the cardiac conduction system (CCS) in failing and infarcted hearts may be the optimal route to heart regeneration. This is because the CCS is vital for generating coordinated contraction and relaxation rhythms of the heart.

Treating failing and infarcted hearts via direct injection of cardiac Purkinje cells into the heart can potentially restore the efficiency of electrical signal conduction and improve cardiac function because cardiac Purkinje cells may intrinsically integrate with the CCS of the recipient's heart. However, current methods of forming cardiac Purkinje cells have numerous limitations in terms of efficiency and reproducibility. Numerous embodiments of the present disclosure address the aforementioned limitations.

### Compositions

In some embodiments, the present disclosure pertains to novel compositions. In some embodiments, the compositions of the present disclosure are suitable for use in differentiating cardiac progenitor cells to cells that resemble cardiac Purkinje cells (herein referred to as cardiac Purkinje-like cells). In some embodiments, the compositions of the present disclosure include the following compounds: (1) Rolipram (i.e., 4-(3-(Cyclopentyloxy)-4-methoxyphenyl)pyrrolidin-2-one), a derivative thereof, a combination thereof; (2) Forskolin (i.e., 3R,4aR,5S,6S,6aS,10S,10aR,10bS)-3-Ethenyl-6,10,10b-trihydroxy-3,4a,7,7,10a-pentamethyl-1-oxododecahydro-1H-naphtho[2,1-b]pyran-5-yl acetate), a derivative thereof, or a combination thereof; (3) CHIR99021 (i.e., 6-((2-((4-(2,4-Dichlorophenyl)-5-(4-methyl-1H-imidazol-2-yl)pyrimidin-2-yl)amino)ethyl)amino)nicotinonitrile), a derivative thereof, or a combination thereof; (4) SB431542 (i.e., 4-[4-(2H-1,3-Benzodioxol-5-yl)-5-(pyridin-2-yl)-1H-imidazol-2-yl]benzamide), a derivative thereof, or a combination thereof; (5) Valproic acid (i.e., 2-propylpentanoic acid), a derivative thereof, or a combination thereof; (6) RG108 (i.e., N-Phthalyl-L-tryptophan), a derivative thereof, or a combination thereof; (7) Parnate (i.e., trans-2-phenylcyclopropylamine), a derivative thereof, or a combination thereof; (8) Resveratrol (i.e., 5-[(E)-2-(4-Hydroxyphenyl) ethen-1-yl]benzene-1,3-diol), a derivative thereof, or a combination thereof; (9) Retinoic acid (i.e., ((2E,4E,6E,8E)-3,7-dimethyl-9-(2,6,6-trimethylcyclohexen-1-yl) nona-2,4,6,8-tetraenoic acid), a derivative thereof, or a combination thereof; and (10) a Neuregulin protein, a derivative thereof, or a combination thereof. In some embodiments, the compositions of the present disclosure also include Sodium Nitroprusside, a derivative thereof, or a combination thereof. In some embodiments, the compositions of the present disclosure also include Epinephrine (i.e., 4-[(1R)-1-hydroxy-2-(methylamino)ethyl]benzene-1,2-diol), a derivative thereof, or a combination thereof.

In some embodiments, the compositions of the present disclosure include one or more derivatives of one or more of the compounds. In some embodiments, the one or more derivatives include one or more moieties derivatized with one or more functional groups. In some embodiments, the one or more functional groups include, without limitation, alkanes, alkenes, ethers, alkynes, alkoxyls, aldehydes, carboxyls, hydroxyls, hydrogens, sulfurs, phenyls, cyclic rings, aromatic rings, heterocyclic rings, linkers, methyl groups, hydrogen groups, tracing agents, derivatives thereof, and combinations thereof.

In some embodiments, the Neuregulin protein includes, without limitation, Neuregulin-1 (SEQ ID NO: 1), Neuregulin-2 (SEQ ID NO: 2), Neuregulin-3 (SEQ ID NO: 3), Neuregulin-4 (SEQ ID NO:4), or combinations thereof. In some embodiments, a derivative of a Neuregulin protein includes a protein that shares at least 60% sequence identity with one or more of Neuregulin-1, Neuregulin-2, Neuregulin-3, and Neuregulin-4. In some embodiments, a derivative of a Neuregulin protein includes a protein that shares at least 70% sequence identity with one or more of Neuregulin-1. Neuregulin-2, Neuregulin-3, and Neuregulin-4. In some embodiments, a derivative of a Neuregulin protein includes a protein that shares at least 80% sequence identity with one or more of Neuregulin-1, Neuregulin-2, Neuregulin-3, and Neuregulin-4. In some embodiments, a derivative of a Neuregulin protein includes a protein that shares at least 90% sequence identity with one or more of Neuregulin-1, Neuregulin-2, Neuregulin-3, and Neuregulin-4. In some embodiments, a derivative of a Neuregulin protein includes a protein that shares at least 95% sequence identity with one or more of Neuregulin-1, Neuregulin-2, Neuregulin-3, and Neuregulin-4.

The compounds in the compositions of the present disclosure can have various concentrations. For instance, in some embodiments, each compound in the compositions of the present disclosure has a concentration ranging from about 100 nM to about 250 μM. In some embodiments, each compound in the compositions of the present disclosure has a concentration ranging from about 1 μM to about 100 μM. In some embodiments, each compound in the compositions of the present disclosure has a concentration ranging from about 1 ng/mL to about 100 ng/mL.

### Methods of Generating Cardiac Purkinje-Like Cells

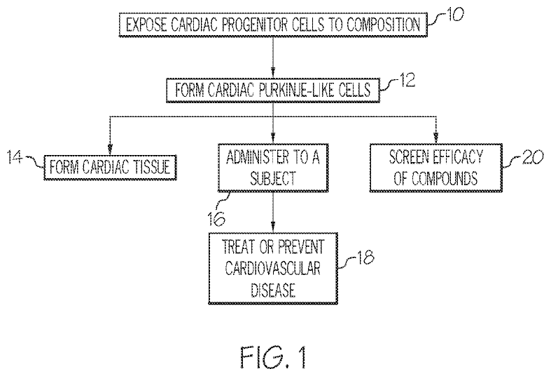

Additional embodiments of the present disclosure pertain to methods of utilizing the compositions of the present disclosure to generate differentiated cardiac cells (i.e., cardiac Purkinje-like cells). In some embodiments illustrated in FIG. 1, the methods of the present disclosure include exposing cardiac progenitor cells to a composition of the present disclosure (step 10). In some embodiments, the exposing results in the differentiation of the cardiac progenitor cells to cardiac Purkinje-like cells (step 12).

As also illustrated in FIG. 1, the methods of the present disclosure can have various diagnostic and therapeutic applications. For instance, in some embodiments, the methods of the present disclosure may utilize the formed cardiac Purkinje-like cells to generate a tissue, such as a cardiac tissue (step 14). In some embodiments, the methods of the present disclosure may utilize the formed cardiac Purkinje-like cells for therapeutic applications, such as through administering the Purkinje-like cells to a subject (step 16) in order to treat or prevent a cardiovascular disease in the subject (step 18). In some embodiments, the methods of the present disclosure may utilize the formed cardiac Purkinje-like cells for diagnostic applications, such as assessing the efficacy of compounds in the treatment or prevention of a cardiovascular disease (step 20).

As set forth in more detail herein, the methods of the present disclosure can have numerous embodiments. In particular, various methods may be utilized to expose various cardiac progenitor cells to various compositions in order to form various cardiac Purkinje-like cells. Additionally, the cardiac Purkinje-like cells may be utilized for various diagnostic and therapeutic applications.

### Cardiac Progenitor Cells

Cardiac progenitor cells generally refer to endogenous cardiac stem cells that are distributed throughout the heart. The methods of the present disclosure may differentiate various types of cardiac progenitor cells. For instance, in some embodiments, the cardiac progenitor cells include, without limitation, adipose mesenchymal stem cells (ADMSC), human adipose mesenchymal stem cells (hADMSC), human induced pluripotent stem cells (iPSCs), cardiac progenitor cell lines, primary cardiac myocytes, and combinations thereof.

In some embodiments, the cardiac progenitor cells include human cardiac progenitor cells. In some embodiments, the human cardiac progenitor cells include cells that have been reprogrammed using ETS2 and MESP1 transcription factors. In some embodiments, the cardiac progenitor cells include human cardiac progenitor cells that have been reprogrammed from human induced pluripotent stem cells using small molecules. In some embodiments, the human cardiac progenitor cells include one or more of the following cell lines: AC16 and HCM.

### Cardiac Purkinje-Like Cells

Cardiac Purkinje-like cells generally refer to differentiated cells that are formed after the exposure of cardiac progenitor cells to one or more of the compositions of the present disclosure. The methods of the present disclosure may be utilized to form various types of cardiac Purkinje-like cells. For instance, in some embodiments, the cardiac Purkinje-like cells are genetically, functionally, morphologically, and electrophysiologically similar to native cardiac Purkinje cells.

In some embodiments, cardiac Purkinje-like cells express genes that are expressed in native cardiac Purkinje cells. In some embodiments, the genes include, without limitation, CNTN2, ETV1, PCP4, IRX3, SCN5a, HCN2, and combinations thereof.

Without being bound by theory, differentiation of cardiac progenitor cells to cardiac Purkinje-like cells after exposure to the compositions of the present disclosure occurs through numerous mechanisms. For instance, in some embodiments, the differentiation occurs by direct reprogramming through transdifferentiation. In some embodiments, the transdifferentiation occurs without a pluripotency state, that is, produced through an epigenetically unstable “plastic” state, where the conversion of fully differentiated and matured cells into a different and novel cell type is facilitated.

### Exposure of Cardiac Progenitor Cells to Compositions

Various methods may also be utilized to expose the compositions of the present disclosure to cardiac progenitor cells in order to form cardiac Purkinje-like cells. For instance, in some embodiments, the exposing occurs in vitro. In some embodiments, the exposing occurs in vivo in a subject.

### Therapeutic Applications

The cardiac Purkinje-like cells that are formed by the methods of the present disclosure can have numerous therapeutic applications. As such, in some embodiments, the methods of the present disclosure also include a step of utilizing the formed cardiac Purkinje-like cells for therapeutic applications.

For instance, in some embodiments, the present disclosure pertains to methods of treating or preventing a cardiovascular disease in a subject by administering one or more compositions of the present disclosure to the subject. Thereafter, the administered compositions facilitate the differentiation of cardiac progenitor cells to cardiac Purkinje-like cells in the subject.

In additional embodiments, the present disclosure pertains to methods of treating or preventing a cardiovascular disease in a subject by administering the pre-formed cardiac Purkinje-like cells of the present disclosure to a subject. The cardiac Purkinje-like cells may be formed by exposing cardiac progenitor cells to one or more compositions of the present disclosure. In some embodiments, the methods of the present disclosure may also include a step of pre-forming the cardiac Purkinje-like cells by exposing cardiac progenitor cells to one or more compositions of the present disclosure.

The cardiac Purkinje-like cells and compositions of the present disclosure may be administered to subjects in various manners. For instance, in some embodiments, the administration includes a local administration of the cardiac Purkinje-like cells or compositions of the present disclosure to a cardiac tissue of the subject. In some embodiments, the cardiac tissue is near or at the ventricular myocardium. In some embodiments, the administration occurs by a method that includes, without limitation, intravenous administration, intramuscular administration, intradermal administration, intraperitoneal administration, subcutaneous administration, spray-based administration, aerosol-based administration, and combinations thereof.

The cardiac Purkinje-like cells and compositions of the present disclosure may be administered to various subjects. For instance, in some embodiments, the subjects include human beings. In some embodiments, the subjects may be suffering from a cardiovascular disease. As such, in some embodiments, the cardiac Purkinje-like cells and compositions of the present disclosure may be utilized to treat the cardiovascular disease.

In some embodiments, the subjects may be vulnerable to a cardiovascular disease. As such, in some embodiments, the cardiac Purkinje-like cells and compositions of the present disclosure may be used to prevent a cardiovascular disease. In some embodiments, the cardiac Purkinje-like cells and compositions of the present disclosure may be used to treat and prevent a cardiovascular disease.

The cardiac Purkinje-like cells and compositions of the present disclosure may be utilized to treat or prevent various types of cardiovascular diseases. For instance, in some embodiments, the cardiovascular disease includes heart failure. In some embodiments, the heart failure includes myocardial infarction (MI)-induced heart failure. In some embodiments, the cardiovascular disease includes arrhythmia. In some embodiments, the arrhythmia includes sudden death resulting from ischemic ventricular tachycardia (VT), hypertrophic and dilated cardiomyopathy including arrhythmogenic right ventricular cardiomyopathy (ARVC), long QT syndrome (LQT), Brugada syndrome, catecholaminergic polymorphic ventricular tachycardia, drug-induced arrhythmias, Lenègre-Lev disease, sick sinus syndrome (SSS), and atrial fibrillation.

Without being bound by theory, the administration of the cardiac Purkinje-like cells and compositions of the present disclosure can treat or prevent cardiovascular diseases in subjects through various mechanisms. For instance, in some embodiments, the administration of the cardiac Purkinje-like cells and compositions of the present disclosure results in cardiac tissue regeneration, cardiac tissue repair, restoration of the cardiac conduction system (CCS), and combinations thereof. In some embodiments, the administration of the cardiac Purkinje-like cells and compositions of the present disclosure results in restoration of the cardiac conduction system (CCS) in the cardiac tissue of a subject. In some embodiments, the administered cardiac Purkinje-like cells and compositions of the present disclosure integrate with the electrical system of the recipient myocardium and propagate electrical impulses in a synchronous manner.

### Applications in Tissue Generation

The cardiac Purkinje-like cells that are formed by the methods of the present disclosure can also have applications in tissue generation. As such, in some embodiments, the methods of the present disclosure also include a step of utilizing the formed cardiac Purkinje-like cells to generate a tissue, such as a cardiac tissue.

In some embodiments, the present disclosure pertains to methods of generating a cardiac tissue by exposing cardiac progenitor cells to one or more compositions of the present disclosure and associating the cardiac progenitor cells with a tissue scaffold. In some embodiments, the association occurs prior to, during, or after the exposing step. In some embodiments, the association occurs prior to the exposing step. In some embodiments, the association occurs during the exposing step. In some embodiments, the association occurs prior to and during the exposing step. In some embodiments, the association occurs after the exposing step. In some embodiments, the association occurs prior to and during the exposing step.

In some embodiments, the exposing results in the differentiation of cardiac progenitor cells to cardiac Purkinje-like cells. In some embodiments, the tissue scaffold is in the form of an artificial heart. In some embodiments, the tissue scaffold includes, without limitation, injectable tissue scaffolds, in situ polymerizable tissue scaffolds, hydrogel-based scaffolds, printable scaffolds (e.g., 3D printable scaffolds), biodegradable composite hydrogel scaffolds, extracellular scaffolds, extracellular matrix-derived nanomaterial-based scaffolds, and combinations thereof.

In some embodiments, the tissue scaffold can be utilized as a carrier for the administration of the purkinje-like cells to a subject. In some embodiments, the tissue scaffold can be utilized for myocardial integration, retention, and synchrony of the Purkinje-like cells in the subject. In some embodiments, the tissue scaffold is in the form of an artificial heart, such as decellularized tissue-based scaffolds, vascularized cardiac patches, personalized hydrogels, and combinations thereof. In some embodiments, the tissue scaffold can be utilized as a bioink for the 3D printing of the tissue scaffold onto various surfaces.

### Diagnostic Applications

The cardiac Purkinje-like cells that are formed by the methods of the present disclosure can also have various diagnostic applications. As such, in some embodiments, the methods of the present disclosure also include a step of utilizing the formed cardiac Purkinje-like cells for diagnostic applications.

In some embodiments, the methods of the present disclosure pertain to methods of assessing the efficacy of one or more compounds in the treatment or prevention of a cardiovascular disease. In some embodiments, such methods include exposing the one or more compounds to the cardiac Purkinje-like cells of the present disclosure; and assessing the efficacy of the one or more compounds in the treatment or prevention of the cardiovascular disease. In some embodiments, the assessing includes observing a change in a property of the cardiac Purkinje-like cells and correlating the change in the property to the efficacy of the one or more compounds in the treatment or prevention of the cardiovascular disease.

Various changes in the properties of cardiac Purkinje-like cells may be observed and correlated to the efficacy of the one or more compounds in the treatment or prevention of a cardiovascular disease. For instance, in some embodiments, the change includes, without limitation, an improvement in ischemia-reperfusion injury, a change in electrical pacing, a change in optical pacing, a change in dysglycemia, a change in oxidative stress, a change in cyclic stretch, and combinations thereof.

In some embodiments, the one or more compounds to be screened include, without limitation, proteins, small molecules, peptides, nucleotides, and combinations thereof. In some embodiments, the one or more compounds to be screened include anti-arrhythmic compounds. As such, in some embodiments, the methods of the present disclosure include assessing the efficacy of anti-arrhythmic compounds in the treatment or prevention of a cardiovascular disease.

In some embodiments, the methods of the present disclosure also include a step of forming the cardiac Purkinje-like cells by exposing cardiac progenitor cells to a composition of the present disclosure.

The cardiac Purkinje-like cells of the present disclosure may be utilized to screen various compounds for their efficacy in the treatment or prevention of various types of cardiovascular diseases. For instance, in some embodiments, the cardiovascular disease includes heart failure. In some embodiments, the heart failure includes myocardial infarction (MI)-induced heart failure. In some embodiments, the cardiovascular disease includes arrhythmia. In some embodiments, the arrhythmia includes sudden death resulting from ischemic ventricular tachycardia (VT), hypertrophic and dilated cardiomyopathy including arrhythmogenic right ventricular cardiomyopathy (ARVC), long QT syndrome (LQT), Brugada syndrome, catecholaminergic polymorphic ventricular tachycardia, drug-induced arrhythmias Lenègre-Lev disease, sick sinus syndrome (SSS), and atrial fibrillation.

In some embodiments, cardiac Purkinje-like cells can be grown in culture systems, such as organ-on-chips to assess the efficacy of drugs in treating or preventing various types of cardiovascular diseases (e.g., arrhythmias).

Many animal models poorly predict the efficacy of drug treatment in humans because some metabolites which can cause systemic toxicity are organism dependent. Therefore, the diagnostic methods of the present disclosure can provide an important alternative to assessing the efficacy of various compounds in treating or preventing cardiovascular diseases.

### Cardiac Purkinje-Like Cells

Additional embodiments of the present disclosure pertain to the cardiac Purkinje-like cells of the present disclosure, which are formed by exposing cardiac progenitor cells to a composition of present disclosure. In some embodiments, the cardiac progenitor cells include, without limitation, adipose mesenchymal stem cells (ADMSC), human adipose mesenchymal stem cells (hADMSC), human induced pluripotent stem cells (iPSCs), cardiac progenitor cell (CPC) lines, primary cardiac myocytes, and combinations thereof. In some embodiments, the cardiac progenitor cells include human cardiac progenitor cells.

In some embodiments, the cardiac Purkinje-like cells resemble cardiac Purkinje cells. In some embodiments, the cardiac Purkinje-like cells are genetically, functionally, morphologically, and electrophysiologically similar to native cardiac Purkinje cells.

### Cardiac Tissues

Additional embodiments of the present disclosure pertain to cardiac tissues that include the cardiac Purkinje-like cells of the present disclosure. In some embodiments, the cardiac tissue is associated with a tissue scaffold. In some embodiments, the tissue scaffold in the form of an artificial heart.

In some embodiments, the cardiac tissue includes a decellularized tissue-based scaffold. In some embodiments, the cardiac tissue is in the form of a vascularized cardiac patch. In some embodiments, the cardiac tissue is in the form of a hydrogel. In some embodiments, the hydrogel is injectable, in situ polymerizable, printable (e.g., 3D printable), and/or biodegradable. In some embodiments, the cardiac tissue is in the form of an extracellular matrix-derived nanomaterial-based bioink, which may be suitable for 3D printing.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Direct Reprogramming of Cardiomyocytes into Cardiac Purkinje-Like Cells

In this Example, Applicant demonstrates an effective protocol of trans-differentiation of human cardiomyocytes (AC16 and iPSC-CM) into cardiac Purkinje-like cells using small molecules. The differentiated Purkinje-like cells exhibit similar transcriptomic profiles and electrophysiological functionality to native Purkinje cells. Transcriptome and immunocytochemistry analysis revealed the expression of key cardiac Purkinje genes such as CNTN2, ETV1, PCP4, IRX3, SCN5a, HCN2, and more. Functional analysis of the Purkinje-like cells demonstrated conduction of electrical signals with increased velocity. Thus, the cells generated were genetically and functionally similar to native cardiac Purkinje cells.

In particular, Applicant generated cardiac Purkinje-like cells for potential treatment of heart failure (HF). Applicant demonstrates in this Example the successful direct differentiation of human cardiomyocyte cell lines (AC16-CMs and iPSC-CMs) into Purkinje-like cells using a unique cocktail of small molecules. Upon treatment of the CMs with a small molecule differentiation cocktail (“PURK-cocktail”), Applicant observed tremendous morphological changes, such that the PURK-cocktail treated cells differentiated and converted to closely morphologically resemble native cardiac Purkinje cells.

Moreover, immunocytochemistry, FACS, and transcriptome analyses demonstrated that Applicant's generated Purkinje-like cells expressed a range of specific cardiac Purkinje genes, such as PCP4, ETV1, CNTN2, NKX2-5, and more. Applicant was also able to demonstrate that the reprogrammed Purkinje-like cells were functionally similar to native cardiac Purkinje cells since they were able to rapidly conduct electrical impulses with a faster conduction velocity compared to the control CMs. Thus, Applicant's reprogrammed Purkinje-like cells displayed morphological, transcriptomic, and functional characteristics similar to that of native cardiac Purkinje cells.

### Example 1.1. Creation of CNTN2-mCherry Reporter Cell Line

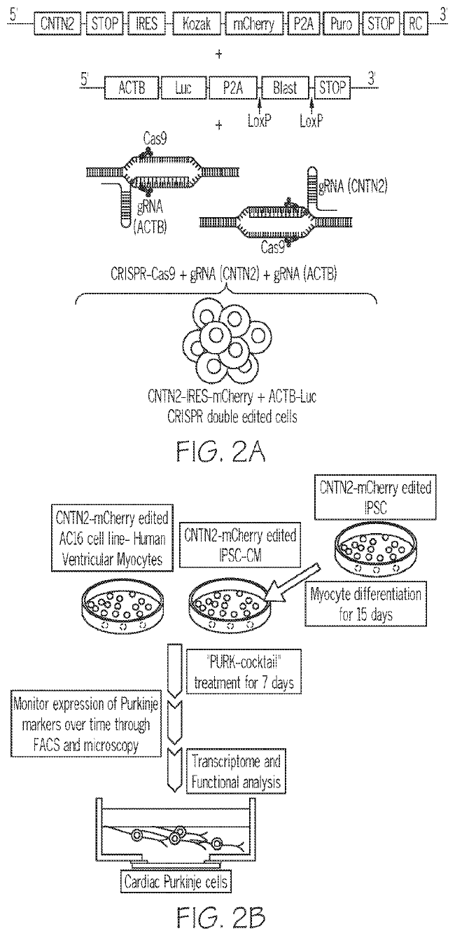

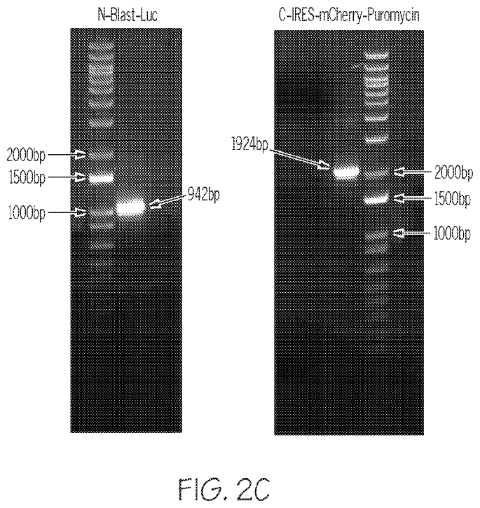

The first step prior to differentiation was to genetically edit the cells so that differentiation could be tracked and monitored by the expression of a specific Purkinje reporter gene. An IRES-mCherry-Puromycin tag was introduced into the Contactin 2 (CNTN2) gene. FIG. 2A depicts the overall approach to creating Applicant's co-gene edited cells.

CNTN2 is an adhesion molecule that can participate in migration, adhesion, neurite outgrowth and fasciculation, myelination, and axon pathfinding during brain development. CNTN2 was identified as a specific marker of cardiac Purkinje cells. Therefore, the expression of CNTN2 was used to indicate whether Applicant's treated cells were differentiating into Purkinje-like cells. To be able to select all of the CRISPR-edited cells, the cells were co-gene edited with a Blasticidin-Luciferase tag on the Beta-actin (ACTB) gene (FIG. 2A). This allowed Applicant to select the edited cells prior to differentiation since mCherry-Puromycin-CNTN2 would only be expressed on differentiated cells.

Upon CRISPR-Cas9 knock-in co-edition of the CNTN2 and ACTB, the edited cells were selected with Blasticidin treatment and genotyped to confirm that the editing was successful in both genes. As shown in FIG. 2C, genotyping confirmed that the cells were successfully edited to express both an IRES-mCherry-Puromycin tag on the CNTN2 gene and a BLAST-Luc tag on the ACTB. This was shown by the PCR products with the exact size of the corresponding tags.

### Example 1.2. Development of the PURK-Cocktail

Several combinations of small molecules were tested. The top combination of a small molecule for the PURK-differentiation cocktail was then identified. The PURK-cocktail is composed of 11 small molecules as shown in Table 1.

TABLE 1
Detailed list of small molecules used in the PURK-cocktail.

| | Final | | Stock | |
|---|---|---|---|---|
| Drug name | Concentration | | Vehicle | Manufacturer |
| Rolipram | 2 | μM | DMSO | SigmaAldrich, |
| | | | | cat# R6520-10MG |
| Forskolin | 10 | μM | DMSO | SigmaAldrich, |
| | | | | cat# F3917-10MG |
| CHIR99021 | 4 | μM | DMSO | SigmaAldrich, |
| | | | | cat# 361571-5MG |
| SB431542 | 2 | μM | DMSO | SigmaAldrich, |
| | | | | cat# 616464-5MG |
| Valproic acid | 2 | μM | Water | SigmaAldrich, |
| | | | | cat# P4543-10G |
| RG108 | 2 | μM | DMSO | SigmaAldrich, |
| | | | | cat# R8279-10MG |
| Parnate | 2 | μM | Water | SigmaAldrich, |
| | | | | cat# 616431-500MG |
| Resveratrol | 10 | μM | DMSO | SigmaAldrich, |
| | | | | cat# R5010-100MG |
| Retinoic Acid | 1 | μM | DMSO | SigmaAldrich, |
| | | | | cat# R2625-500MG |
| Neuregulin | 10 | ng/mL | Water | StemCell Technologies, |
| | | | | cat# 78071.1 |
| Epinephrine | 10 | μM | 0.05M | SigmaAldrich, |
| | | | HCl | cat# E4250-5G |

### Example 1.3. Treatment with the PURK-Cocktail Provokes Differentiation

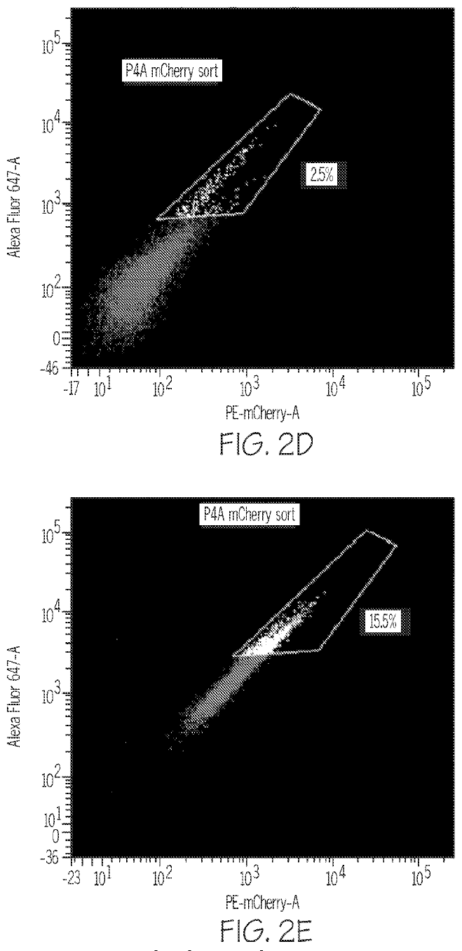

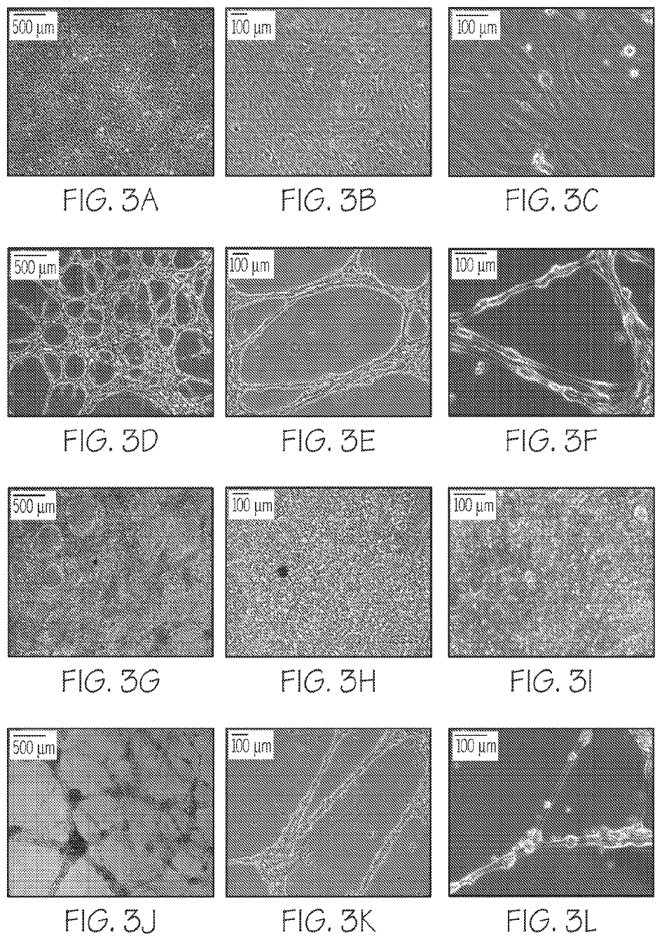

Applicant tested various small molecule drug combinations. For the purposes of this Example, Applicant decided to focus on the data obtained from the PURK-cocktail. The overall scheme for differentiating the cardiomyocytes (CMs) into Purkinje-like cells using Applicant's small molecule differentiation cocktail (“PURK-cocktail”) is shown in FIG. 2B. Upon treatment of the AC16 and iPSC-CM cells with the PURK-cocktail, tremendous morphological changes were observed (FIGS. 3D-3F and FIGS. 3J-3L) compared to the control cells (FIGS. 3A-C and FIGS. 3G-I). The cells treated with the PURK-differentiation cocktail displayed a Purkinje-like morphology, where they had round bodies and prolonged projections (FIGS. 3F and 3L). Moreover, it could be observed that the differentiating cells were highly adherent to one another and organized themselves in a “net-like” network (FIGS. 3D and 3J).

Since the cells were grown and differentiated in a thick Fibrin matrix, it was observed that the cells freely moved within the Fibrin matrix as they differentiated, and their network was three-dimensional (3D). No apparent morphological changes were observed in the vehicle-treated control cells, despite them also growing on a Fibrin matrix. The iPSC-CMs were functionally active and were able to beat without extra stimulation upon transfer into a Fibrin matrix coated dish. The extreme changes in cell morphology were observed as early as day-2 of differentiation. Additionally, the cells maintained their morphological profile, even after the removal of treatment. The differentiating cells also noticeably stopped proliferating or proliferated very slowly compared to the control.

Additionally, the differentiating cells samples were analyzed via Fluorescence Activated Cell Sorting (FACS) (FIGS. 2D-E). A small group of CNTN2-mCherry+ cells in the PURK-cocktail treatment was observed. It was observed that the optimal cell sorting day was day-7, where the highest number of CNTN2-mCherry+ cells could be obtained via FACS. As shown in FIG. 2E, the percentage of CNTN2-mCherry+ was 15.5% for the “PURK-cocktail”-treated cells, compared to control-treated cells (FIG. 2D), which showed a minimal amount of CNTN2-mCherry+ cells (2.5%, P<0.005). All collected cells were then used for downstream experiments.

### Example 1.4. The Differentiated Cells Express Purkinje Specific Genes

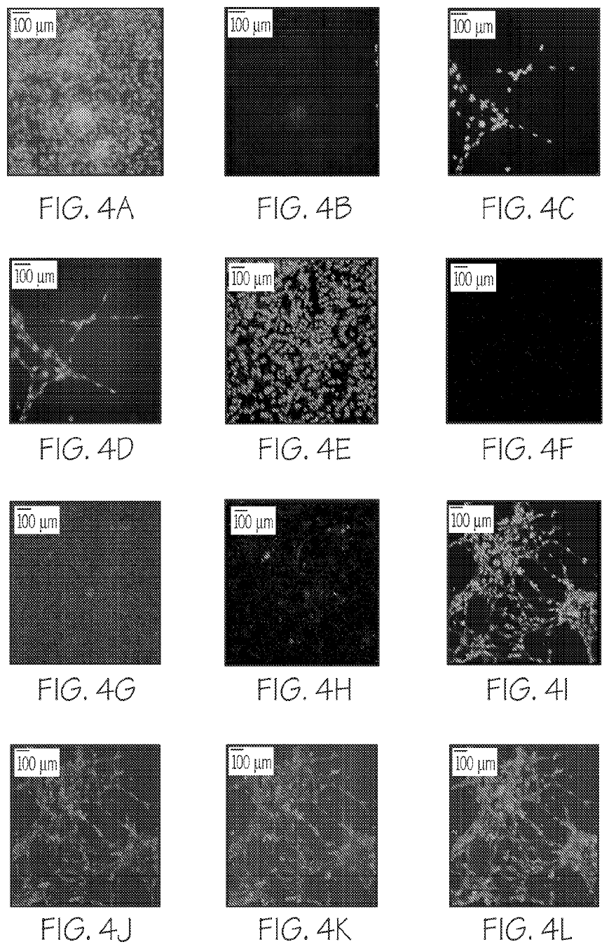

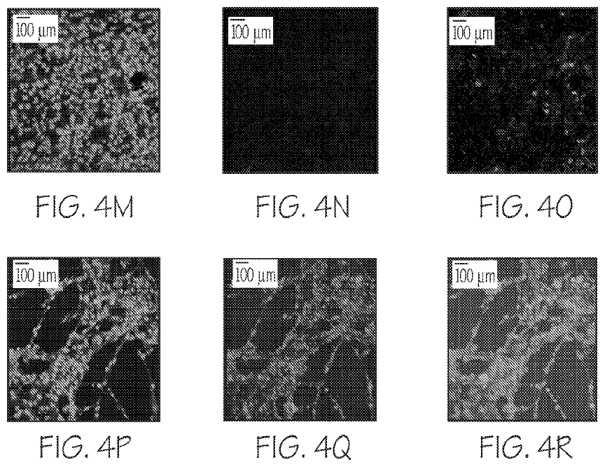

Next, to characterize the PURK-cocktail treated cells and determine whether they expressed key cardiac Purkinje markers, Applicant performed immunocytochemistry. Antibodies against ETV1, SCN5a, PCP4, and IRX3 were used. Additionally, the expression of the CRISPR-Cas9 knock-in CNTN2-IRES-mCherry in the samples was also evaluated through fluorescent microscopy (FIG. 4). The control cells did not show any signal for the Purkinje specific markers and did not express the CNTN2-IRES-mCherry reporter gene (FIGS. 4B, 4F, 4G, 4H, and 4O). On the other hand, the PURK-cocktail treated cells were stained for all Purkinje specific markers and highly expressed the CNTN2-IRES-mCherry reporter gene (FIGS. 4D, 4J, 4K, 4L, 4Q, and 4R). SCN5a, IRX3, and PCP4 were shown to be expressed in the cell membrane of all differentiated cells. On the other hand, ETV1, a nuclear Purkinje cell marker, was shown to be expressed in the cells' nucleus. The CNTN2-IRES-mCherry reporter was seen in both the cell nucleus and cell surface.

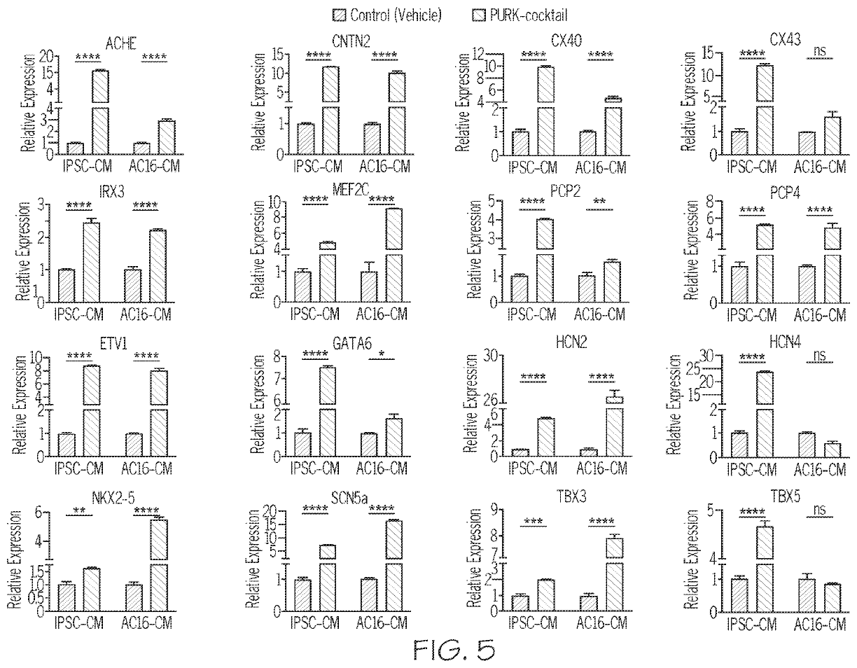

To further characterize the differentiated cells, the expression of 16 cardiac Purkinje-specific genes was analyzed through qRT-PCR. As shown in FIG. 5, the PURK-cocktail induced a different transcriptomic profile in both AC16-CMs and iPSC-CMs compared to the control. In the PURK-cocktail treated AC16 cells, 13 of the 16 genes evaluated were significantly upregulated (P<0.05, n=3). HCN4 and TBX5 appeared to be downregulated; however, the difference was not statistically significant. In the PURK-cocktail treated iPSC-CMs, all 16 genes evaluated were significantly upregulated. The expression of key neuronal Purkinje markers was also evaluated. No expression of TUBB3, LHX5, SKOR2, and OLIG2 was found in any of the samples (data not shown).

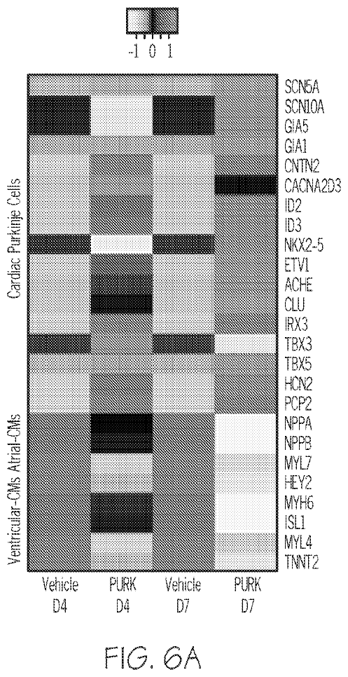

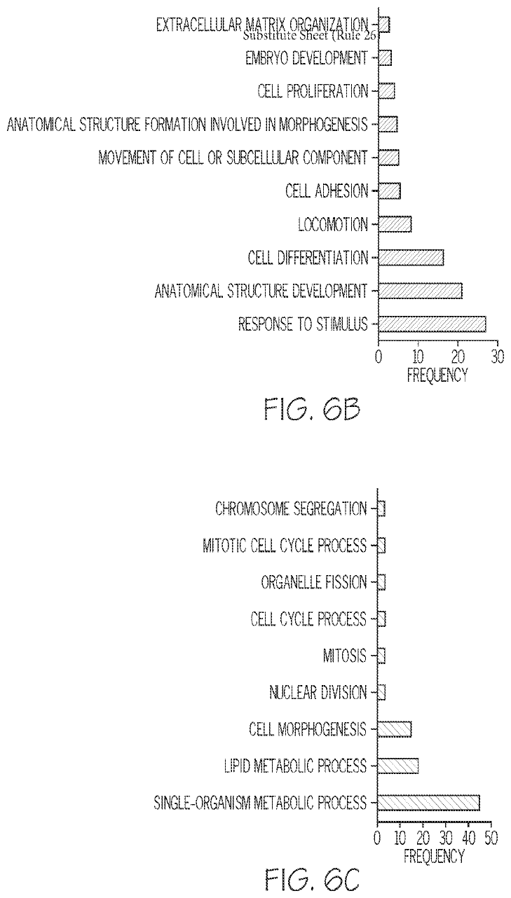

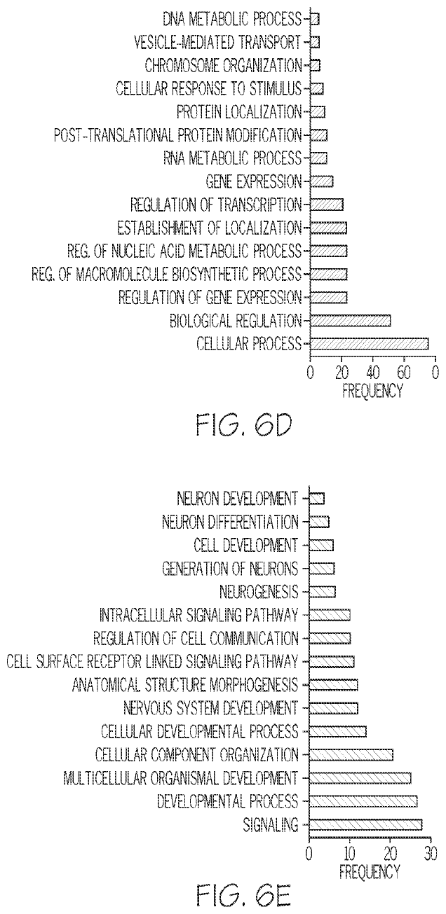

RNA-seq was then used to further evaluate the cells' transcriptome. As shown in FIG. 5A, key cardiac Purkinje genes were identified with significant expression changes (P<0.05). Day-4 and day-7 differentiation samples showed slightly different expressions of these genes, with day-7 showing the most robust cardiac Purkinje gene expression profile. The data also shows a down-regulation of ventricular and atrial myocyte-specific genes. Furthermore, the Gene Ontology (GO) analysis shown in FIGS. 6B-E suggests a major difference in the genes and pathways that are up- and down-regulated between day-4 and day-7 of differentiation.

### Example 1.5. The Purkinje-Like Cells are Functionally Similar to Native Purkinje Cells

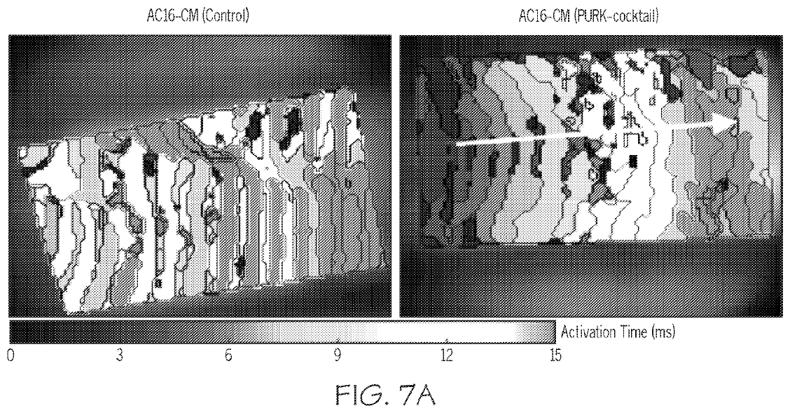

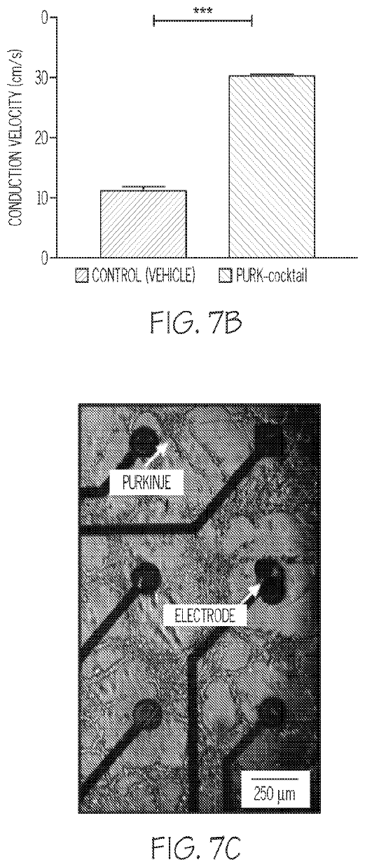

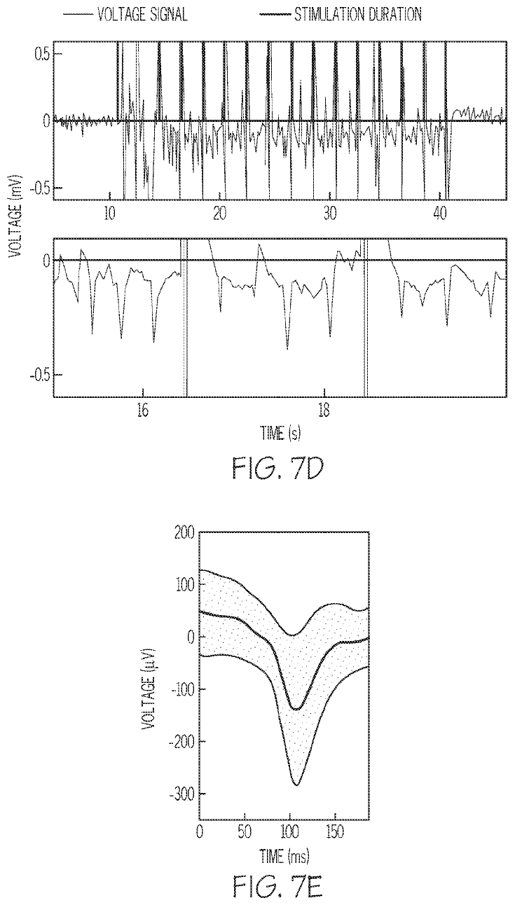

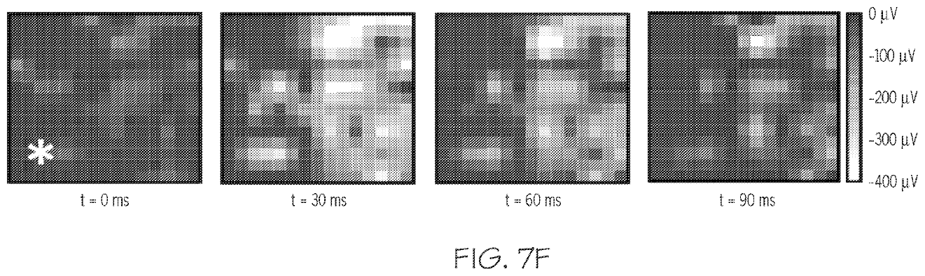

Applicant's next step was to characterize the function of the differentiated cells via optical mapping and multielectrode array (MEA) electrophysiological studies. Based on optical mapping studies, the activation map of the control cells revealed a very slow pulsating-like activation from the cells, rather than conduction of the electrical stimulation (FIG. 7A). In contrast, the activation map of the PURK-cocktail treated cells showed that the cells were rapidly activated upon electrical stimulation and produced a very strong electrical signal propagation throughout the whole dish (FIG. 7A). The conduction velocity (CV) was almost 3-times faster in the PURK-cocktail treated cells than in the control cells (FIG. 7B).

Moreover, the PURK-cocktail treated cells can be seen on the MEA after 7 days in culture (FIG. 7C). The projections extend over a span of a few millimeters and are fully connected with the other Purkinje-like cells throughout the entirety of the MEA surface area (approximately 50 mm²). Electrophysiological recordings were performed to visualize the activity of the Purkinje-like cells. Electrical stimulation was applied through two of the electrodes on the MEA to stimulate cells. The single-channel recording (FIG. 7D) shows that prior to the beginning of the stimulation (first spike in red), no electrical activity was evident. However, after the first few stimulation pulses, the Purkinje-like cells responded by emitting multiple pulse waves in a row (FIG. 7D). After the stimulation concluded, no more electrical activities were observed.

A single spike was isolated in the time domain and averaged across all 64 channels of the MEA. The average of the pulse waveform is shown in black in FIG. 7E, and the gray area indicates the standard deviation of the signal. The heatmaps in FIG. 7F demonstrate the spatial activations of the PURK-cocktail treated cells across the MEA over time and indicate that the stimulation signal propagated from the bottom left corner of the MEA to a majority of the cells in the network. Control (vehicle) treated cells were not functionally active.

### Example 1.6. Discussion

In this Example, Applicant presented evidence of the generation of cardiac Purkinje-like cells by applying a unique small molecule cocktail (“PURK-cocktail”) to human cardiomyocytes. The Purkinje-like cells created exhibit a similar transcriptomic profile and electrophysiological functionality as native Purkinje cells. In conclusion, this Example demonstrates an innovative way to generate human Purkinje-like cells through direct cell differentiation using a unique small molecule cocktail. Applicant's cocktail may be utilized to directly differentiate clinically relevant cells into Purkinje-like cells. The functionality of the Purkinje-like cells is further verified by a collective characterization, including electrical stimulation, optical mapping, and MEA recording.

Accordingly, this Example may facilitate to advance the quest in finding an optimized cell therapy that can aid in heart regeneration, potentially being further translated into the clinical setting in the upcoming years. Furthermore, the cells generated in this Example may be essential for tissue engineering artificial heart models in vitro. Moreover, an in vitro heart model has the potential to be used for the development and investigation of new pharmacological therapies for heart diseases.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A composition comprising the following compounds:
Rolipram (4-(3-(Cyclopentyloxy)-4-methoxyphenyl)pyrrolidin-2-one), a derivative thereof, or a combination thereof;
Forskolin (3R,4aR,5S,6S,6aS,10S,10aR,10bS)-3-Ethenyl-6,10,10b-trihydroxy-3,4a, 7,7,10a-pentamethyl-1-oxododecahydro-1H-naphtho[2,1-b]pyran-5-yl acetate), a derivative thereof, or a combination thereof;
CHIR99021 (6-((2-((4-(2,4-Dichlorophenyl)-5-(4-methyl-1H-imidazol-2-yl)pyrimidin-2-yl)amino)ethyl)amino)nicotinonitrile)), a derivative thereof, or a combination thereof;
SB431542 (4-[4-(2H-1,3-Benzodioxol-5-yl)-5-(pyridin-2-yl)-1H-imidazol-2-yl]benzamide), a derivative thereof, or a combination thereof;
Valproic acid (2-propylpentanoic acid), a derivative thereof, or a combination thereof;
RG108 (N-Phthalyl-L-tryptophan), a derivative thereof, or a combination thereof;
Parnate (trans-2-phenylcyclopropylamine), a derivative thereof, or a combination thereof;
Resveratrol (5-[(E)-2-(4-Hydroxyphenyl) ethen-1-yl]benzene-1,3-diol), a derivative thereof, or a combination thereof;
Retinoic acid ((2E,4E,6E,8E)-3,7-dimethyl-9-(2,6,6-trimethylcyclohexen-1-yl) nona-2,4,6,8-tetraenoic acid), a derivative thereof, or a combination thereof; and
a Neuregulin protein, a derivative thereof, or a combination thereof.

2. The composition of claim 1, wherein the composition further comprises Sodium Nitroprusside, a derivative thereof, or a combination thereof.

3. The composition of claim 1, wherein the composition further comprises Epinephrine (4-[(1R)-1-hydroxy-2-(methylamino)ethyl]benzene-1,2-diol), a derivative thereof, or a combination thereof.

4. The composition of claim 1, wherein the composition comprises one or more derivatives of one or more of the compounds, wherein the one or more derivatives comprise one or more moieties derivatized with one or more functional groups, wherein the one or more functional groups is selected from the group consisting of alkanes, alkenes, ethers, alkynes, alkoxyls, aldehydes, carboxyls, hydroxyls, hydrogens, sulfurs, phenyls, cyclic rings, aromatic rings, heterocyclic rings, linkers, methyl groups, hydrogen groups, tracing agents, derivatives thereof, and combinations thereof.

5. (canceled)

6. The composition of claim 1, wherein each compound has a concentration ranging from about 100 nM to about 250 μM.

7-8. (canceled)

9. A method of generating differentiated cardiac cells, said method comprising:
exposing cardiac progenitor cells to a composition of comprising the following compounds:
Rolipram (4-(3-(Cyclopentyloxy)-4-methoxyphenyl)pyrrolidin-2-one), a derivative thereof, or a combination thereof;
Forskolin (3R,4aR,5S,6S,6aS,10S,10aR,10bS)-3-Ethenyl-6,10,10b-trihydroxy-3,4a,7,7,10a-pentamethyl-1-oxododecahydro-1H-naphtho[2,1-b]pyran-5-yl acetate), a derivative thereof, or a combination thereof;
CHIR99021 (6-((2-((4-(2,4-Dichlorophenyl)-5-(4-methyl-1H-imidazol-2-yl)pyrimidin-2-yl)amino)ethyl)amino)nicotinonitrile), a derivative thereof, or a combination thereof;
SB431542 (4-[4-(2H-1,3-Benzodioxol-5-yl)-5-(pyridin-2-yl)-1H-imidazol-2-yl]benzamide), a derivative thereof, or a combination thereof;
Valproic acid (2-propylpentanoic acid), a derivative thereof, or a combination thereof;
RG108 (N-Phthalyl-L-tryptophan), a derivative thereof, or a combination thereof;
Parnate (trans-2-phenylcyclopropylamine), a derivative thereof, or a combination thereof;
Resveratrol (5-[(E)-2-(4-Hydroxyphenyl) ethen-1-yl]benzene-1,3-diol), a derivative thereof, or a combination thereof;
Retinoic acid ((2E,4E,6E,8E)-3,7-dimethyl-9-(2,6,6-trimethylcyclohexen-1-yl) nona-2,4,6,8-tetraenoic acid), a derivative thereof, or a combination thereof; and
a Neuregulin protein, a derivative thereof, or a combination thereof.

10. The method of claim 9, wherein the cardiac progenitor cells are selected from the group consisting of adipose mesenchymal stem cells (ADMSC), human adipose mesenchymal stem cells (hADMSC), human induced pluripotent stem cells (iPSCs), cardiac progenitor cell (CPC) lines, primary cardiac myocytes, human cardiac progenitor cells, and combinations thereof.

11. (canceled)

12. The method of claim 9, wherein the exposing results in the differentiation of the cardiac progenitor cells to cells that resemble cardiac Purkinje cells, and wherein the differentiated cardiac cells are genetically, functionally, morphologically, and electrophysiologically similar to native cardiac Purkinje cells.

13. The method of claim 9, wherein the exposing occurs in vivo in a subject.

14. The method of claim 9, wherein the exposing occurs in vitro.

15. The method of claim 13, wherein the exposing comprises administering the composition of claim 9 to the subject, and wherein the A method is used to treat or prevent a cardiovascular disease in a subject.

16. The method of claim 15, wherein the administering comprises locally administering the composition to a cardiac tissue of the subject.

17. The method of claim 16, wherein the cardiac tissue is near or at the ventricular myocardium.

18. The method of claim 15, wherein the subject is suffering from the cardiovascular disease, and wherein the method is used to treat the cardiovascular disease.

19. The method of claim 15, wherein the cardiovascular disease comprises heart failure (HF), arrhythmia, or combinations thereof.

21-21. (canceled)

22. A method of treating or preventing a cardiovascular disease in a subject, said method comprising:
administering differentiated cardiac cells to a subject,
wherein the differentiated cardiac cells are formed by exposing cardiac progenitor cells to a composition comprising the following compounds:
Rolipram (4-(3-(Cyclopentyloxy)-4-methoxyphenyl)pyrrolidin-2-one), a derivative thereof, or a combination thereof;
Forskolin (3R,4aR,5S,6S,6aS,10S,10aR,10bS)-3-Ethenyl-6,10,10b-trihydroxy-3,4a,7,7,10a-pentamethyl-1-oxododecahydro-1H-naphtho[2,1-b]pyran-5-yl acetate), a derivative thereof, or a combination thereof;
CHIR99021 (6-((2-((4-(2,4-Dichlorophenyl)-5-(4-methyl-1H-imidazol-2-yl)pyrimidin-2-yl)amino)ethyl)amino)nicotinonitrile)), a derivative thereof, or a combination thereof;
SB431542 (4-[4-(2H-1,3-Benzodioxol-5-yl)-5-(pyridin-2-yl)-1H-imidazol-2-yl]benzamide), a derivative thereof, or a combination thereof;
Valproic acid (2-propylpentanoic acid), a derivative thereof, or a combination thereof;
RG108 (N-Phthalyl-L-tryptophan), a derivative thereof, or a combination thereof;
Parnate (trans-2-phenylcyclopropylamine), a derivative thereof, or a combination thereof;
Resveratrol (5-[(E)-2-(4-Hydroxyphenyl) ethen-1-yl]benzene-1,3-diol), a derivative thereof, or a combination thereof;
Retinoic acid ((2E,4E,6E,8E)-3,7-dimethyl-9-(2,6,6-trimethylcyclohexen-1-yl) nona-2,4,6,8-tetraenoic acid), a derivative thereof, or a combination thereof; and
a Neuregulin protein, a derivative thereof, or a combination thereof.

23. The method of claim 22, further comprising a step of forming the differentiated cardiac cells, wherein the forming comprises exposing cardiac progenitor cells to the composition of claim 22.

24. The method of claim 22, wherein the administering comprises locally administering the differentiated cardiac cells to a cardiac tissue of the subject.

25. The method of claim 24, wherein the cardiac tissue is near or at the ventricular myocardium.

26. (canceled)

27. The method of claim 22, wherein the cardiovascular disease comprises heart failure (HF), arrhythmia, or combinations thereof.

29-29. (canceled)

30. The method of claim 14, wherein the method is used to generate a cardiac tissue, and wherein the method further comprises a step of
associating the cardiac progenitor cells with a tissue scaffold.

31. The method of claim 30, wherein the associating occurs prior to, during, or after the exposing step.

32. The method of claim 30, wherein the tissue scaffold in the form of an artificial heart.

43-43. (canceled)
